(19) United States
(12) Patent Application Publication
(10) Pub. No.: US 2006/0292103 A1
(43) Pub. Date: Dec. 28, 2006

(54) Composition for skin use

(75) Inventors: Ming-Hung Huang, Changhua County (TW); Po-Han Chen, Tainan City (TW); Ying-Fei Huang, Tainan City (TW); Ya-Ping Hung, Tainan City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Taiyen Biotech. Co., LTd.

(21) Appl. No.: 11/304,176

(22) Filed: Dec. 15, 2005

(30) Foreign Application Priority Data
Jun. 24, 2005 (TW) 94121282

(51) Int. Cl. A61K 8/64; A61K 8/37

(52) U.S. Cl. 424/70.14; 424/70.31

(57) ABSTRACT

A composition for skin use is disclosed. The composition for skin use comprises a high concentration of protein fiber dispersed in an emulsifier system that is consisted of at least one non-silicone surfactant. The emulsifier system has no silicone-containing component, so as to improve the disadvantages of the prior emulsifier system for applying to the material of cosmetics or external medicines. In another embodiment of the present invention, the composition for skin use further comprises the high concentration of protein fibers dispersed in a wax and an emulsifier system that is consisted of at least one non-silicone surfactant.

### RELATED APPLICATIONS

The present application is based on, and claims priority from, Taiwan Application Serial Number 94121282, filed Jun. 24, 2005, the disclosure of which is hereby incorporated by reference herein in its entirety.

### FIELD OF THE INVENTION

The present invention relates to a composition for use on skin, and more particularly, to a composition for use on skin comprising an emulsifier system including at least one non-silicone surfactant.

### BACKGROUND OF THE INVENTION

Collagen is the most important constituent of extracellular matrix and also a major structural protein of animal connective tissue. Collagen exists mainly in the form of an insoluble fibrillar protein. In the constitution of human body, collagen has an inseparable relation with organ, tissue and cell, and can be used in repair and regeneration of human organ and tissue.

Since collagen makes up 70% of the human dermis, collagen is very useful for skin. Firstly, collagen is capable of retaining moisture thereby strengthening the water-retaining function of skin, and plays an important role in helping the connective tissue of the skin to retain moisture. Secondly, collagen makes up about one-third of total cell protein. The protein in the dermis is mainly composed of collagen fiber and elastic fiber. The content of collagen affects the skin's resilience and luster. Furthermore, through the interaction effect of retaining water and encouraging firmness, collagen can improve secreting situation and promote pore contraction of oily skin as well as retain moisture of dry skin thereby transforming skin into neutral skin with firmness and fine texture. Collagen can repair damaged skin such as acne and wrinkles.

Collagen is a major constituent for maintaining the elasticity of skin and muscle. As age increases, skin and muscle begin to age due to loss of moisture therein. In the meanwhile, collagen fiber becomes thinner and thinner, and the elasticity of elastic fiber decreases. The regular mesh structure constituted by collagen and elastin gradually collapses thereby resulting in wrinkles, loose skin, darkness, fleck and roughness. The collagen of skin may be replenished through diet, cosmetics or topical medicine.

Typically, the cosmetic industry may be divided into several categories, including cleansing, skin care, make up, fragranced, hair care, medicine cosmetics, etc, of which fragranced, hair care and medicine cosmetics are the main stream of cosmetic sales. The effectiveness of a cosmetic product depends on its ingredients, its manufacturing process and the person using the cosmetic product. Due to the progress in the modern cosmetic technology, the damage of cosmetics to the skin is minimized. Some cosmetic products are added with skin-care ingredients to provide skin care function.

Generally speaking, when collagen is applied in a cosmetic product with skin-care function, it is dissolved in a water phase solution and then mixed with other ingredients of the cosmetic product. Although there are many different types of collagen, only water-insoluble collagen can be applied in cosmetic products. Furthermore, the concentration of the water-insoluble collagen in the water phase solution is limited to not more than 1 weight percent. This significantly increases the manufacturing time and steps of the cosmetic products.

In addition, the ingredients of cosmetic products typically include cosmetic base materials, surfactants and cosmetic additives such as fragrances, pigments, binders, antioxidants and antibacterials, wherein careful selection of the cosmetic base materials is critical. The base typically include oil phase composition such as vegetable oil, mineral oil, fatty acid, wax, and synthetic ester, water phase composition and emulsifiers. However, some kinds of fatty acids in the base, e.g., capric acid, oleyl alcohol or some synthetic esters (such as isopropyl isostearate), can easily permeate into skin, and in turn will irritate skin and promote acne. Since the base contains emulsifiers, the phenomenon of flaky powder is observed after a long period of time.

Recently, because silicon-containing surfactants (such as silicone) are not easily absorbed by skin and cause less damage to skin, they are used to replace synthetic esters and emulsifiers as the base of cosmetic products in order to reduce the problems mentioned above. Although silicon-containing surfactants give slippery feeling, they clog pores easily. It is impossible to wash away the silicon-containing surfactants thoroughly because of their water-insoluble property. Therefore, the residual silicon-containing surfactants will accumulate in the pores as the number of times the cosmetic products is used increases, thereby clogging the pores and suffocating the skin.

Accordingly, there is a need for a composition for use on skin which improves upon the drawbacks associated with the conventional emulsifier system.

### SUMMARY OF THE INVENTION

It is an object of the present invention to provide a composition for skin use including a high concentration of protein fiber evenly dispersed in an emulsifier system that is consisted of at least one non-silicone surfactant. The emulsifier system has no silicone-containing component thereby reducing the disadvantages of the conventional emulsifier system such that the composition can be used as the material of cosmetics or topical medicines.

Another object of the present invention is to provide a composition for use on skin including a high concentration of protein fiber dispersed in a wax and an emulsifier system comprised of at least one non-silicone surfactant, which can be used as the material of cosmetics or topical medicines.

According to the above and other objects of the present invention, there is provided a composition for use on skin comprising a protein fiber evenly dispersed in an emulsifier system including at least one non-silicone surfactant represented by the formula (I):
Xₘ₁—O—[Cₘ₂—O—]ₙHₘ₃ (I)

wherein:

X is selected from the group consisting of C8-C24 fatty acid carbonyl group (C7-CO- to C23-CO-), C8-C24 pareth group [(C8-O-)ₚC8- to (C24-O-)ₚC24-], C8-C24polyglyceryl alkylate group [C7-COO—(CH₂CH₂OHCH₂—)_{q} to C23-COO—(CH₂CH₂OHCH₂—)_{q}], and the derivatives thereof; m1 is 1 or 2; m2 is 2 or 3; m3 is 0 or 1; m1 plus m3 equals 2; n is an integer of from 10 to 100; p is an integer of from 1 to 19; q is an integer of from 1 to 3.

According to one embodiment of the present invention, the content of the protein fiber may be from 0.5 to 40 weight percent based on the total weight of the composition.

According to one embodiment of the present invention, the protein fiber includes but is not limited to collagen, hydrolyzed collagen, water-soluble collagen, and water-insoluble collagen.

According to one embodiment of the present invention, the content of the emulsifier system may be from 0.1 to 90 weight percent based on the total weight of the composition.

According to one embodiment of the present invention, the fatty acid includes but is not limited to monofatty acid, difatty acid, and the derivatives thereof.

According to one embodiment of the present invention, the fatty alcohol includes but is not limited to monofatty alcohol, difatty alcohol ether, and the derivatives thereof.

According to one embodiment of the present invention, the aforementioned composition may be formulated as cosmetics or topical medicines.

According to the above and other objects of the present invention, there is provided another composition for use on skin comprising a wax, an emulsifier system evenly mixed with the wax, and a protein fiber evenly dispersed in the emulsifier system. The emulsifier system includes at least one non-silicone surfactant represented by the formula (I):
Xₘ₁—O—[Cₘ₂—O—]ₙHₘ₃ (I)

wherein:

X is selected from the group consisting of C8-C24 fatty acid carbonyl group (C7-CO- to C23-CO-), C8-C24 pareth group [(C8-O-)ₚC8- to (C24-O-)ₚC24-], C8-C24polyglyceryl alkylate group [C7-COO—(CH₂CH₂OHCH₂—)_{q} to C23-COO—(CH₂CH₂OHCH₂—)_{q}], and the derivatives thereof; m1 is 1 or 2; m2 is 2 or 3; m3 is 0 or 1; m1 plus m3 equals 2; n is an integer of from 10 to 100; p is an integer of from 1 to 19; q is an integer of from 1 to 3.

According to one embodiment of the present invention, the content of the wax is from 10 to 90 weight percent based on the total weight of the composition.

According to one embodiment of the present invention, the aforementioned composition may be formulated as cosmetics or topical medicines, and the content of the wax, the emulsifier system and the protein fiber is from 1 to 100 weight percent based on the total weight of the composition.

The composition for use on skin according the present invention adopts an emulsifier system having no silicone-containing component to reduce the disadvantages of the conventional emulsifier system such that the composition can be used as the material of cosmetics or topical medicines.

### DETAILED DESCRIPTION OF THE PREFERRED EMBODIMENTS

The present invention is directed to a composition for skin use including protein fiber evenly dispersed in an emulsifier system that is consisted of at least one non-silicone surfactant such that the composition can be used as the material of cosmetics or topical medicines. The composition for skin use is described in detail below.

Specifically, the emulsifier system of the present invention is consisted of at least one non-silicone surfactant represented by the formula (I):
Xₘ₁—O—[Cₘ₂—O—]ₙHₘ₃ (I)

wherein:

X is selected from the group consisting of C8-C24 fatty acid carbonyl group (C7-CO- to C23-CO-), C8-C24 pareth group [(C8-O—)ₚC8- to (C24-O—)ₚC24-], C8-C24polyglyceryl alkylate group [C7-COO—(CH₂CH₂OHCH₂—)_{q} to C23-COO—(CH₂CH₂OHCH₂—)_{q}], and the derivatives thereof; m1 is 1 or 2; m2 is 2 or 3; m3 is 0 or 1; m1 plus m3 equals 2; n is an integer of from 10 to 100; p is an integer of from 1 to 19; q is an integer of from 1 to 3.

The non-silicone surfactant is exemplified by, but not limited to, polyethylene glycol(PEG)-alkylates, PEG-pareth, PEG-polyglyceryl alkylates, polypropylene glycol(PPG)-alkylates, PPG-pareth, and PPG-polyglyceryl alkylates. According to one embodiment of the present invention, the polyethylene glycol or polypropylene glycol preferably has a low degree of polymerization, e.g., ranged between 10 and 100. The fatty acid is preferably a C8-C24 fatty acid such as lauric acid (C12), myristic acid (C14), palmitic acid (C16), stearic acid (C18). The fatty alcohol is preferably a C8-C24fatty alcohol. The fatty alcohol includes but is not limited to monofatty alcohol, difatty alcohol ether, and the derivatives thereof. The present invention utilizes the aforementioned non-silicone surfactant to form the emulsifier system thereby reducing the irritation and burden to skin, and significantly increasing the concentration of the protein fiber in the composition for use on skin. The phenomenon of flaky powder is not observed in the application of cosmetics made from the composition of the present invention.

The protein fiber of the present invention includes but is not limited to collagen, hydrolyzed collagen, water-soluble collagen, and water-insoluble collagen. The collagen of the present invention is mainly utilized as a moisture-retaining and skin-caring ingredient thereby providing skin-caring function in the application of the composition for use on skin.

In one embodiment, the composition for use on skin of the present invention includes protein fiber evenly dispersed in the emulsifier system wherein the content of the protein fiber may be from 0.5 to 40 weight percent, and the content of the emulsifier system may be from 0.1 to 90 weight percent, based on the total weight of the composition. In another embodiment, the composition of the present invention further includes a wax such that the emulsifier system is evenly mixed with the wax and the protein fiber is evenly dispersed in the emulsifier system, wherein the content of the protein fiber may be from 0.5 to 40 weight percent, and the content of the emulsifier system may be from 0.1 to 80 weight percent, and the content of the wax is from 10 to 90 weight percent, based on the total weight of the composition.

According to one embodiment of the present invention, the composition for use on skin may be used as the material of cosmetics or topical medicines and in combination with topical ingredients of the cosmetics or topical medicines. When the composition is formulated as cosmetics or topical medicines, and the content of the wax, the emulsifier system and the protein fiber is from 1 to 100 weight percent based on the total weight of the composition. Suitable topical ingredients include, but not limited to, whitening agents, moisture-retaining agents, antioxidants, UV absorbents, surfactants, thickeners, dyes, skin nutriments, etc.

While the composition of the present invention is formulated as cosmetics or topical medicines, it may be incorporated with whitening agents and/or other active ingredients, e.g., vitamine C, arbutin, Kojic Acid, tyrosinase inhibitors such as quercetin and catechin, anti-acne agents, anti-bacteria agents, analgesic agents, anaesthetics, anti-inflammatory agents, antipruritic agents, antihyperkeratolytic agents, anti-dry skin agents, antipsoriatic agents, anti-aging agents, antiwrinkle agents, antiseborrheic agents, self-tanning agents, wound-healing agents, corticosteroids or hormones.

Furthermore, while the composition of the present invention is formulated as cosmetics or topical medicines, it may further comprise adjuvants or other ingredients. Suitable adjuvants include, but not limited to, hydrophilic or hydrophobic gelling agents, hydrophilic or hydrophobic active agents, preserving agents, antioxidants, solvents, fragrances, fillers, screening agents, chelating agents, odor absorbers and dyes. Other ingredients include, but not limited to, carbohydrates such as lactose and glucose, excipients (such as glucose, cellulose, silicate, and calcium carbonate), binders (such as cellulose, starch, gelatin, carbohydrates) and disintegrants (such as agar powder, gelatin powder, sodium alginate, cellulose derivatives and calcium carbonate). The content of the adjuvants or ingredients mentioned above in the composition can be easily determined by those skilled in the art and is therefore not described in detail herein.

The composition of the present invention may be formulated as cosmetics or topical medicines in the form of oil-in-water, water-in-oil, water-in-oil-in-water or oil-in-water-in-oil emulsions, cream, ointment, milk, lotion, serum, paste or dispersion. In addition, the composition of the present invention may be formulated as cosmetics in the form of lip colors, foundations, milk, cream, mask, gel, aerosol, milky lotions, mousse, dispersion, cream, packs and wash-away cleansing milks or wash soap.

Examples of the composition for use on skin will be given below, but the present invention should not be construed as being limited thereto.

### EXAMPLE 1

To 1650 g water phase liquid was added 680 g doughlike water-insoluble collagen, and then added 550 g oil phase liquid containing 90% of isohexadecane. After the resulting solution was stirred well, an emulsifier system (115 g) was added thereto thereby obtaining an evenly emulsified product that can be used as the material of cosmetics. The emulsifier system contains PEG-30 dipolyhydroxystearate (about 60 g) and PPG-15 stearyl ether (about 55 g).

### EXAMPLE 2

340 g hydrolyzed collagen water solution was evenly dispersed in 600 g wax and an emulsifier system (120 g) containing about 30 g to about 60 g of olive oil PEG-40 glycerol ester and about 60 g to about 90 g of PPG-20 oleyl ether thereby obtaining a product that can be used as the material of cosmetics.

### EXAMPLE 3

To 1200 g water phase liquid was added 500 g doughlike water-insoluble collagen, and then added 550 g oil phase liquid containing 90% of squalene. After the resulting solution was stirred well, an emulsifier system (145 g) was added thereto thereby obtaining an evenly emulsified product that can be used as the material of topical medicines. The emulsifier system contains about 30 g to about 50 g of PEG-30 dipolyhydroxystearate and about 45 g to about 65 g of PPG-15 stearyl ether.

### EXAMPLE 4

550 g oil phase liquid containing 90% of squalene was added to 1000 g hydrolyzed collagen water solution. After the resulting solution was stirred well, an emulsifier system (140 g) was added thereto thereby obtaining a product that can be used as the material of topical medicines. The emulsifier system contains about 35 g to about 70 g of PPG-4 ceteareth-12 and about 70 g to about 105 g of PEG-40 glyceryl cocoate.

Briefly, the composition for use on skin of the present invention is characterized by having a high concentration of protein fiber evenly dispersed in an emulsifier system consisted of at least one non-silicone surfactant. In contrast, the concentration of the water-insoluble collagen in the conventional cosmetics or topical medicines is limited to not more than 1 weight percent of water-insoluble collagen, since only a low concentration of water-insoluble collagen can be dissolved in a water phase solution and then mixed with other ingredients of the conventional cosmetics or topical medicines in the conventional manufacturing process. The composition for use on skin of the present invention is also characterized by having an emulsifier system that can be used to dissolve all kinds of collagen, such as collagen, hydrolyzed collagen, water-soluble collagen, and water-insoluble collagen, to a high concentration of from 0.5 to 40 weight percent while simplifying the manufacturing steps of cosmetics or topical medicines; in the meanwhile, the time required for manufacturing cosmetics or topical medicines is reduced and the manufacturing steps are simplified. In addition, the emulsifier system of the present invention has no silicone-containing component thereby reducing the disadvantages of the conventional emulsifier system such that the composition can be used as the material of cosmetics or topical medicines.

According to the aforementioned preferred embodiments, one advantage of the composition for use on skin of the present invention is that a high concentration of protein fiber is evenly dispersed in an emulsifier system consisted of at least one non-silicone surfactant or in a wax and an emulsifier system consisted of at least one non-silicone surfactant. The emulsifier system of the present invention has no silicone-containing component thereby reducing the disadvantages of the conventional emulsifier system such that the composition can be used as the material of cosmetics or topical medicines.

As is understood by a person skilled in the art, the foregoing preferred embodiments of the present invention are illustrated of the present invention rather than limiting of the present invention. It is intended to cover various modifications and similar arrangements included within the spirit and scope of the appended claims, the scope of which should be accorded the broadest interpretation so as to encompass all such modifications and similar structure.

What is claimed is:

1. A composition for use on skin, comprising:
an emulsifier system including at least one non-silicone surfactant represented by the formula (I):
Xₘ₁—O—[Cₘ₂—O—]ₙHₘ₃ (I)
wherein:
X is selected from the group consisting of C8-C24 fatty acid carbonyl group (C7-CO- to C23-CO-), C8-C24 pareth group [(C8-O—)ₚC8- to (C24-O—)ₚC24-], polyglyceryl C8-C24 alkylate group [C7-COO—(CH₂CH₂OHCH₂—)_{q} to C23-COO—(CH₂CH₂OHCH₂—)_{q}], and the derivatives thereof; m1 is 1 or 2; m2 is 2 or 3; m3 is 0 or 1; m1 plus m3 equals 2; n is an integer of from 10 to 100; p is an integer of from 1 to 19; q is an integer of from 1 to 3; and
a protein fiber evenly dispersed in the emulsifier system.

2. The composition according to claim 1, wherein the content of the protein fiber is from 0.5 to 40 weight percent based on the total weight of the composition.

3. The composition according to claim 1, wherein the protein fiber is selected from the group consisting of collagen, hydrolyzed collagen, water-soluble collagen, and water-insoluble collagen.

4. The composition according to claim 1, wherein the content of the emulsifier system is from 0.1 to 90 weight percent based on the total weight of the composition.

5. The composition according to claim 1, wherein the composition is formulated as cosmetics or a topical medicine.

6. The composition according to claim 5, wherein the content of the emulsifier system and the protein fiber is from 1 to 100 weight percent based on the total weight of the composition.

7. The composition according to claim 1, further comprising a wax evenly mixed with the emulsifier system.

8. The composition according to claim 7, wherein the content of the protein fiber is from 0.5 to 40 weight percent based on the total weight of the composition.

9. The composition according to claim 7, wherein the content of the emulsifier system is from 0.1 to 80 weight percent based on the total weight of the composition.

10. The composition according to claim 7, wherein the content of the wax is from 10 to 90 weight percent based on the total weight of the composition.

11. The composition according to claim 7, wherein the composition is formulated as cosmetics or a topical medicine.

12. The composition according to claim 11, wherein the content of the wax, the emulsifier system and the protein fiber is from 1 to 100 weight percent based on the total weight of the composition.

13. The composition according to claim 1, wherein the non-silicone surfactant comprises a PEG-alkylate.

14. The composition according to claim 1, wherein the non-silicone surfactant comprises a PEG C8-C24 pareth.

15. The composition according to claim 1, wherein the non-silicone surfactant comprises a PEG-polyglyceryl alkylate.

16. The composition according to claim 1, wherein the non-silicone surfactant comprises a PPG-alkylate.

17. The composition according to claim 1, wherein the non-silicone surfactant comprises a PPG C8-C24 PARETH.

18. The composition according to claim 1, wherein the non-silicone surfactant comprises a PPG-polyglyceryl alkylate.
